(19) United States
(12) Patent Application Publication
(10) Pub. No.: US 2024/0402107 A1
(43) Pub. Date: Dec. 5, 2024

(54) THERMAL CONDUCTIVITY SENSORS AND ASSOCIATED OPERATING METHODS

(71) Applicant: Infineon Technologies AG, Neubiberg (DE)

(72) Inventor: Ilie-Ionut CRISTEA, Bucharest (RO)

(21) Appl. No.: 18/674,115

(22) Filed: May 24, 2024

(30) Foreign Application Priority Data
Jun. 5, 2023 (DE) 102023205225.3

(51) Int. Cl. G01N 25/18; G01N 33/00; G01R 19/10

(52) U.S. Cl. CPC G01N 25/18; G01N 33/0027; G01R 19/10

(57) ABSTRACT

A thermal conductivity sensor includes a measurement circuit configured to operate in a first circuit configuration during a heating phase of the measurement circuit and in a second circuit configuration during a measurement phase of the measurement circuit. The first circuit configuration is associated with a first power dissipation of the measurement circuit at a supply voltage. The second circuit configuration is associated with a second power dissipation of the measurement circuit at the supply voltage. The first power dissipation is greater than the second power dissipation.

### CROSS-REFERENCE TO RELATED APPLICATION

This application claims priority to German Patent Application No. 102023205225.3 filed on Jun. 5, 2023, the content of which is incorporated by reference herein in its entirety.

### TECHNICAL FIELD

The present disclosure relates to thermal conductivity sensors and methods for operating thermal conductivity sensors.

### BACKGROUND

Thermal conductivity sensors may e.g., be used in the automotive sector or a variety of industrial applications. The sensitivity of a thermal conductivity sensor may inter alia depend on the magnitude of an applied supply voltage. Providing a required minimum sensitivity of the sensor may therefore become problematic for applications in which only a low supply voltage is provided. Manufacturers and designers of thermal conductivity sensors are constantly striving to improve their products. In particular, it may be desirable to provide thermal conductivity sensors with enhanced sensitivity in order to obtain reliable and accurate measurement results even at low supply voltages. In addition, it may be desirable to provide suitable methods for operating such thermal conductivity sensors.

### SUMMARY

An aspect of the present disclosure relates to a thermal conductivity sensor. The thermal conductivity sensor includes a measurement circuit configured to operate in a first circuit configuration during a heating phase of the measurement circuit and in a second circuit configuration during a measurement phase of the measurement circuit. The first circuit configuration is associated with a first power dissipation of the measurement circuit at a supply voltage. The second circuit configuration is associated with a second power dissipation of the measurement circuit at the supply voltage. The first power dissipation is greater than the second power dissipation.

A further aspect of the present disclosure relates to a method for operating a thermal conductivity sensor. The method includes applying a supply voltage to a measurement circuit of the thermal conductivity sensor. The method further includes performing a heating phase of the measurement circuit in a first circuit configuration of the measurement circuit, wherein the first circuit configuration is associated with a first power dissipation of the measurement circuit at the supply voltage. The method further includes performing a measurement phase of the measurement circuit in a second circuit configuration of the measurement circuit, wherein the second circuit configuration is associated with a second power dissipation of the measurement circuit at the supply voltage. The first power dissipation is greater than the second power dissipation.

### BRIEF DESCRIPTION OF THE DRAWINGS

Devices and methods in accordance with the disclosure will be explained in more detail below based on the drawings. Like reference numerals designate corresponding similar parts. The features of the various illustrated examples can be combined unless they exclude each other and/or can be selectively omitted if not described to be necessarily required. Examples are depicted in the drawings and are exemplarily detailed in the description which follows.

FIG. 1 illustrates a circuit diagram of a measurement circuit that may be included in a thermal conductivity sensor.

FIG. 2 illustrates a circuit diagram of a measurement circuit that may be included in a thermal conductivity sensor.

FIGS. 3A-3B illustrate timing diagrams for a temperature and an output voltage of a measurement circuit that may be included in a thermal conductivity sensor.

FIG. 4 illustrates a circuit diagram of a measurement circuit that may be included in a thermal conductivity sensor in accordance with the disclosure.

FIGS. 5A-5B illustrate circuit diagrams of a first circuit configuration and a second circuit configuration of the measurement circuit of FIG. 4.

FIG. 6 illustrates a flowchart of a method for operating a thermal conductivity sensor in accordance with the disclosure.

FIGS. 7A-7B illustrate timing diagrams for a temperature and an output voltage of different measurement circuits.

### DETAILED DESCRIPTION

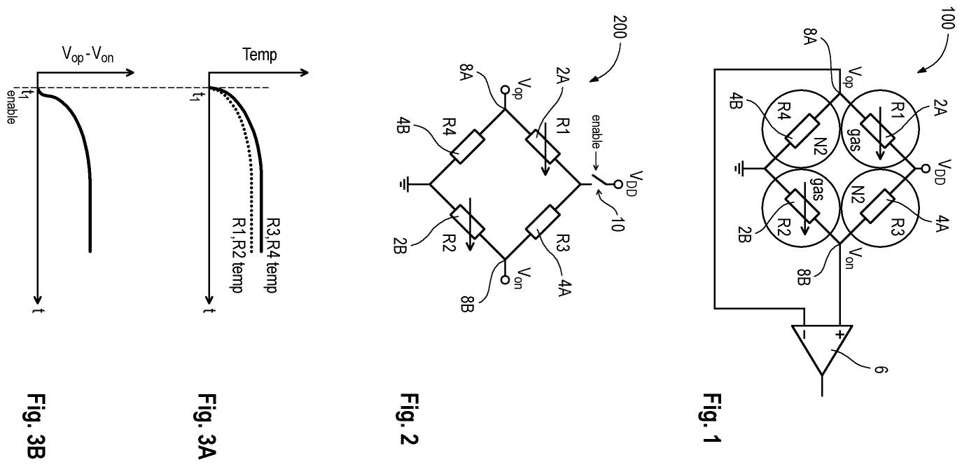

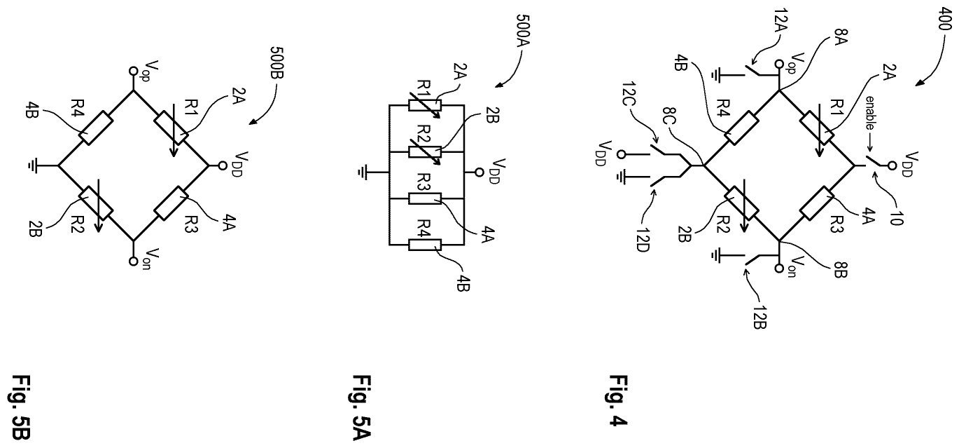

FIG. 1 illustrates a circuit diagram of a measurement circuit 100 that may be included in a thermal conductivity sensor. An operation of the measurement circuit 100 will be discussed later on in connection with FIGS. 3A and 3B. The measurement circuit 100 may include a Wheatstone bridge circuit with two resistors 2A, 2B configured to be exposed to an analysis gas (or a gas of interest) and two resistors 4A, 4B exposed to a reference gas. For example, the analysis gas may correspond to or may include hydrogen or helium, while the reference gas may correspond to or may include nitrogen. The resistors 2A, 2B may be referred to as sensor resistors, while the resistors 4A, 4B may be referred to as reference resistors. Resistance values R1, R2 of the sensor resistors 2A, 2B may change based on the presence and concentration of the analysis gas, while resistance values R3, R4 of the reference resistors 4A, 4B may substantially remain constant.

For example, each of the illustrated resistors may include a hot wire (or heating wire) that may be exposed to the respective gas. The resistors may e.g., be manufactured based on a MEMS (Microelectromechanical systems) technology. In particular, the resistors may be PTC (Positive Temperature Coefficient) resistors configured to conduct electric currents better at low temperatures than at high temperatures. That is, a resistance value of the resistors may be smaller at low temperatures than at high temperatures. As a result, an electric current through a resistor may be a measure for the thermal conductivity of the gas surrounding the resistor.

The measurement circuit 100 may include a component 6 for providing a measurement value specifying a thermal conductivity of the analysis gas. In particular, the component 6 may be configured output a voltage difference Vₒₚ-Vₒₙ measured across the measurement circuit 100 between a first node 8A and a second node 8B. The first node 8A may be arranged between the first sensor resistor 2A and the second reference resistor 4B, while the second node 8B may be arranged between the second sensor resistor 2B and the first reference resistor 4A. In the illustrated example, the component 6 may correspond to or may include a differential amplifier.

FIG. 2 illustrates a circuit diagram of a measurement circuit 200 that may be included in a thermal conductivity sensor. The measurement circuit 200 may be at least partially similar to the measurement circuit 100 of FIG. 1. For the sake of simplicity, the analysis gas and the reference gas as well as the component 6 of FIG. 1 are not shown in FIG. 2. The measurement circuit 200 may include a switch 10 configured to connect and/or disconnect the measurement circuit 200 to a supply voltage V_{DD}.

FIG. 3 includes FIGS. 3A and 3B illustrating timing diagrams for a temperature and an output voltage of a measurement circuit that may be included in a thermal conductivity sensor. For example, the measurement circuit may be similar to the measurement circuit 200 of FIG. 2.

During an operation of the thermal conductivity sensor including the measurement circuit 200, the switch 10 may be closed at a time t₁, for example in response to an enable signal. As a result, the supply voltage V_{DD} may be applied to the measurement circuit 200 and a heating phase of the measurement circuit 200 may be initiated at time t₁. Referring to the timing diagram of FIG. 3A, the resistors of the measurement circuit 200 may heat up during the heating phase until characteristic stable (self-heating) temperatures depending on the thermal conductivity of the respective gas may be reached. The temperature may stabilize to a value equal to the dissipated power divided by the thermal conductivity.

The thermal conductivity of the analysis gas (e.g., hydrogen) may be greater than the thermal conductivity of the reference gas (e.g., nitrogen), such that the characteristic stable temperature of the sensor resistors 2A, 2B (see R1, R2) exposed to the analysis gas may be smaller than the stable characteristic temperature of the reference resistors 4A, 4B (see R3, R4) exposed to the reference gas. When the stable characteristic temperatures are reached, the resistance values R1, R2 of the sensor resistors 2A, 2B may be smaller than the resistance values R3, R4 of the reference resistors 4A, 4B. Due to the different resistance values, the bridge circuit 200 may output a non-zero output voltage difference Vₒₚ-Vₒₙ as shown in the timing diagram of FIG. 3B. The voltage difference Vₒₚ-Vₒₙ may specify the thermal conductivity of the analysis gas relative to the reference gas. Based on the provided output voltage Vₒₚ-Vₒₙ the analysis gas and/or a concentration of the analysis gas may be determined.

FIG. 4 illustrates a circuit diagram of a measurement circuit 400 that may be included in a thermal conductivity sensor in accordance with the disclosure. The measurement circuit 400 may include components as already discussed in connection with foregoing Figs. such that previous comments may also hold true for FIG. 4.

The measurement circuit 400 may include a first switch 12A configured to connect the first node 8A to a ground potential and/or to disconnect the first node 8A from the ground potential. A second switch 12B of the measurement circuit 400 may be configured to connect the second node 8B to a ground potential and/or to disconnect the second node 8B from the ground potential. The measurement circuit 400 may further include at least one third switch configured to selectively connect a third node 8C arranged between the second sensor resistor 2B and the second reference resistor 4B to a ground potential or the supply voltage V_{DD}. In the illustrated example, the at least one third switch may exemplarily include two switches 12C and 12D for providing the connection of the third node 8C to the ground potential or the supply voltage V_{DD}. The discussed switches of the measurement circuit 400 may be activated, deactivated and controlled by any suitable control unit which is not illustrated for the sake of simplicity.

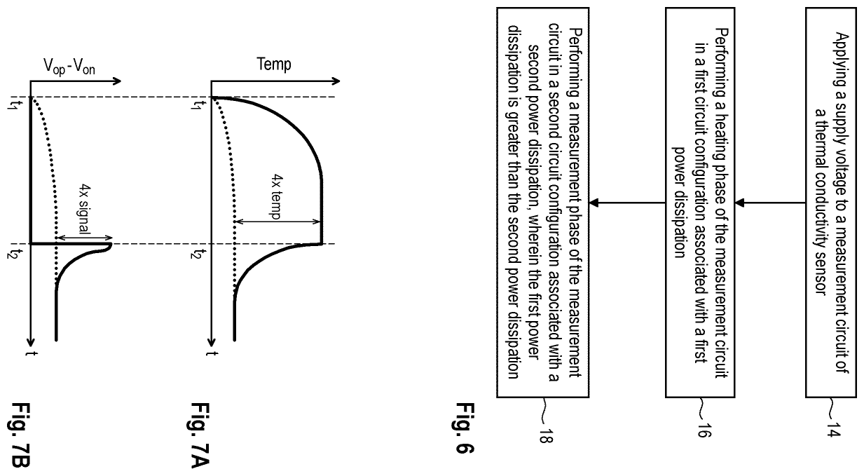

Depending on the positions of the switches 12A to 12D, the measurement circuit 400 may be configured to operate in two different circuit configurations. In this regard, FIGS. 5A and 5B illustrate circuit diagrams of a first circuit configuration 500A and a second circuit configuration 500A of the measurement circuit 400. In both cases, the switch 10 may be closed for providing a connection to the supply voltage V_{DD}. The switches 12A to 12D represent switching means configured to switch the measurement circuit 400 between the first circuit configuration 500A and the second circuit configuration 500B.

In the first circuit configuration 500A of FIG. 5A the switches 12A and 12B may be closed. That is, each of the first node 8A and the second node 8B may be connected to ground potential. In addition, switch 12C may be closed and switch 12D may be open, such that the third node 8C may be connected to the supply voltage V_{DD}. In the first circuit configuration 500A the four resistors 2A, 2B, 4A and 4B may be connected in parallel as shown in FIG. 5A. For illustrative purposes, the resistors of FIG. 4 have been rearranged in FIG. 5A to demonstrate their parallel arrangement. The switches of FIG. 4 are not illustrated in FIG. 5A for the sake of simplicity.

in

In the second circuit configuration 500B of FIG. 5B the switches 12A and 12B may be open. That is, each of the first node 8A and the second node 8B may be disconnected from ground potential. In addition, switch 12C may be open and switch 12D may be closed, such that the third node 8C may be connected to ground potential. In the second circuit configuration 500B the four resistors 2A, 2B, 4A and 4B may be part of a Wheatstone bridge circuit. Accordingly, the second circuit configuration 500B of the measurement circuit 400 may be similar to the measurement circuits 100 and 200 of FIGS. 1 and 2. Similar to FIG. 1, the measurement circuit 400 may include a component for measuring a voltage difference Vₒₚ-Vₒₙ across the measurement circuit 400 in the second circuit configuration 500B, wherein the voltage difference Vₒₚ-Vₒₙ may be indicative of a thermal conductivity of the analysis gas. Again, the switches of FIG. 4 are not illustrated in FIG. 5B for the sake of simplicity.

The circuit configurations 500A and 500B may be connected to the supply voltage V_{DD} during operation as shown in FIGS. 5A and 5B. As a result, electric currents will flow through the resistors in the respective circuit configuration, resulting in power dissipation (and thus self-heating) of the respective circuit configuration. Here, the first circuit configuration 500A may be associated with a first power dissipation of the measurement circuit 400 at the supply voltage V_{DD}, while the second circuit configuration 500B may be associated with a second power dissipation of the measurement circuit 400 at the same supply voltage V_{DD}. Since a parallel arrangement of resistors may increase the amount of dissipated power compared to a serial arrangement of resistors, the first power dissipation of the first circuit configuration 500A may be greater than the second power dissipation of the second circuit configuration 500B. It is noted that the second power dissipation of the second configuration 500B may be similar to a power dissipation of the measurement circuits 100 and 200 of FIGS. 1 and 2.

In the non-limiting illustrated example, the increased power dissipation of the first circuit configuration 500A may be provided by arranging all four resistors in parallel. A number of four parallel resistors may result in the first power dissipation being about four times higher than the second power dissipation. However, in general, providing increased power dissipation in the first circuit configuration 500A may be provided by connecting at least two resistors of the plurality of resistors in parallel. That is, in further examples, a different number of resistors may be connected in parallel.

FIG. 6 illustrates a flowchart of a method for operating a thermal conductivity sensor in accordance with the disclosure. The method is described in a general manner to qualitatively specify aspects of the disclosure. The method may include further aspects. For example, the method may be extended by any of the aspects described in connection with the timing diagrams of FIGS. 7A and 7B discussed later on.

At 14, a supply voltage may be applied to a measurement circuit of a thermal conductivity sensor. At 16, a heating phase of the measurement circuit may be performed in a first circuit configuration of the measurement circuit. The first circuit configuration may be associated with a first power dissipation of the measurement circuit at the supply voltage. At 18, a measurement phase of the measurement circuit may be performed in a second circuit configuration of the measurement circuit. The second circuit configuration may be associated with a second power dissipation of the measurement circuit at the supply voltage. The first power dissipation may be greater than the second power dissipation.

FIGS. 7A and 7B illustrate timing diagrams for a temperature and an output voltage of different measurement circuits. In this regard, dotted curves illustrate timing diagrams for a measurement circuit similar to FIGS. 1 and 2. The dotted curves may therefore be similar to the curves shown in FIGS. 3A and 3B. Furthermore, solid curves illustrate timing diagrams for a measurement circuit similar to the measurement circuit 400 of FIG. 4. The solid curves may be read in connection with the method of FIG. 6 and will be discussed in the following.

Referring back to step 14 of FIG. 6, a supply voltage V_{DD} may be applied to the measurement circuit 400, for example by closing switch 10 in response to an enable signal. The supply voltage V_{DD} may be applied during a time interval between times t₁ and t₂. During this time interval the measurement circuit 400 may be in the first circuit configuration 500A of FIG. 5A. Due to the applied supply voltage V_{DD} a heating phase of the first circuit configuration 500A may be initiated. The heating phase may start at time t₁ and may end at time t₂.

Referring back to step 16 of FIG. 6 and as shown in the timing diagram of FIG. 7A, the measurement circuit 400 being in the first circuit configuration 500A may heat up during the heating phase until a characteristic stable temperature may be reached. As previously described, due to a parallel arrangement of at least two resistors, the first power dissipation of the first circuit configuration 500A may be greater than the power dissipation of the measurement circuit 200 of FIG. 2. Therefore, the characteristic stable temperature of the first circuit configuration 500A may be higher than the characteristic stable temperature of the measurement circuit 200 as can be seen from a comparison between the dotted curve and the solid curve in FIG. 7A. In the illustrated example, the characteristic stable temperature of the first circuit configuration 500A may be about four times higher than the characteristic stable temperature of the measurement circuit 200.

As can be seen from the timing diagram of FIG. 7B, the voltage difference Vₒₚ-Vₒₙ output by the first configuration 500A may be (substantially) zero during the heating phase between times t₁ and t₂. That is, no voltage may be measured across the measurement circuit 400 when being in the first circuit configuration 500A during the heating phase.

When the measurement circuit 400 has reached the characteristic stable temperature, the measurement circuit 400 may be switched from the first circuit configuration 500A to the second circuit configuration 500B at time t₂. That is, the switches 12A to 12C may be opened, and the switch 12D may connect the node 8C to ground potential. As can be seen from the timing diagram of FIG. 7B, switching the measurement circuit 400 to the second circuit configuration 500B may result in a non-zero output voltage difference Vₒₚ-Vₒₙ at time t₂.

Switching the measurement circuit 400 from the first circuit configuration 500A to the second circuit configuration 500B may trigger a measurement for obtaining a measurement value. The measurement may be performed (substantially) at time t₂ (or immediately after time t₂). Referring back to step 18 of FIG. 6, a time interval starting at time t₂ may thus be referred to as measurement phase. Note that the supply voltage V_{DD} may be kept substantially constant during the heating phase and the measurement phase. A measurement at time t₂ may be performed by measuring the voltage difference Vₒₚ-Vₒₙ across the measurement circuit 400 in the second circuit configuration 500B.

As previously discussed with regard to FIG. 7A, the characteristic stable temperature of the first circuit configuration 500A may be higher than the characteristic stable temperature of the measurement circuit 200. Since the measured voltage difference Vₒₚ-Vₒₙ may be proportional to the stable self-heating temperature, the voltage difference Vₒₚ-Vₒₙ at time t₂ for the measurement circuit 400 may be higher than the voltage difference Vₒₚ-Vₒₙ at time t₂ for the measurement circuit 200. This can be seen from a comparison between the dotted curve and the solid curve at time t₂ as shown in FIG. 7B. In the illustrated example, the voltage difference Vₒₚ-Vₒₙ at time t₂ for the measurement circuit 400 may be about four times higher than the voltage difference Vₒₚ-Vₒₙ at time t₂ for the measurement circuit 200.

A higher voltage difference Vₒₚ-Vₒₙ may result in an enhanced measurement sensitivity of the respective thermal conductivity sensor. Therefore, separating the heating phase and the measurement phase based on the two different circuit configurations 500A and 500B may provide enhanced measurement sensitivity compared to the sensitivity of the measurement circuit 200. In the illustrated example, the measurement sensitivity may be increased by a factor of about four. The measurement circuit 400 may thus be configured to provide reliable and accurate measurements even for applications in which only a low supply voltage is provided, while the measurement circuit 200 may fail to provide suitable measurement values in such applications.

In a further step, the analysis gas and/or a concentration of the analysis gas may be detected based on the measured voltage difference Vₒₚ-Vₒₙ. After switching the measurement circuit 400 from the first circuit configuration 500A to the second circuit configuration 500B, the solid curves of FIGS. 7A and 7B asymptotically approach the dotted curves for times greater than t₂.

The previously described examples were based on Wheatstone bridge circuits. However, it is to be noted that the described Wheatstone bridge circuits are example and may be replaced by any other half bridge circuit or bridge circuit configured to provide a measurement value specifying a thermal conductivity of an analysis gas. Accordingly, measurement circuits as described herein may generally correspond to or may include a bridge circuit or a half bridge circuit. Thermal conductivity sensors as described herein are not restricted to the example Wheatstone bridge circuits described herein.

Measurement circuits as described herein may also be referred to as measurement elements. In this regard, it is to be noted that thermal conductivity sensors as described herein may include further circuit components such as e.g., a switch, a signal amplifier, an analog digital converter, etc. However, such components may not necessarily be regarded as part of a measurement circuit as described herein. An operation of these additional components may not necessarily depend on the present temperature of the circuit. In contrast to this, an operation of a bridge circuit and the value of the output voltage may be sensitive to the temperature of the bridge circuit.

Thermal conductivity sensors (or thermal conductivity gas sensors) in accordance with the disclosure may particularly be used as hydrogen sensors for detecting hydrogen and/or hydrogen concentrations. Hydrogen sensors may be used in a variety of applications, such as e.g., in the automotive sector or industrial applications. By way of example, hydrogen sensors may be used for hydrogen exhaust gas detection, exhaust gas monitoring, battery monitoring, hydrogen leakage detection, hydrogen detection in industrial plants, etc.

With a view to achieving climate targets, the automotive industry is promoting and developing the production of hydrogen-powered vehicles. Fuel cell cars can be considered as a breakthrough for electromobility and can heavily contribute to a reduced CO₂ emission. Thermal conductivity sensors as described herein improve hydrogen technology and may thus at least partially contribute to achieving climate targets that have been set. As previously described, thermal conductivity sensors in accordance with the disclosure may provide sensitivities similar to conventional sensors, but at lower supply voltages. This may result in a reduced power consumption of the sensor and therefore a reduced overall power consumption of the respective application including the sensor. The thermal conductivity sensors as described herein save resources and may contribute to energy savings. As a whole, improved thermal conductivity sensors in accordance with the disclosure and methods for operating such sensors may contribute to green technology and green power solutions, e.g., climate-friendly solutions providing reduced energy usage.

ASPECTS In the following, thermal conductivity sensors in accordance with the disclosure and methods for operating such thermal conductivity sensors will be explained using aspects.

Aspect 1 is a thermal conductivity sensor, comprising: a measurement circuit configured to operate in a first circuit configuration during a heating phase of the measurement circuit and in a second circuit configuration during a measurement phase of the measurement circuit, wherein: the first circuit configuration is associated with a first power dissipation of the measurement circuit at a supply voltage, the second circuit configuration is associated with a second power dissipation of the measurement circuit at the supply voltage, and the first power dissipation is greater than the second power dissipation.

Aspect 2 is a thermal conductivity sensor of Aspect 1, wherein: the measurement circuit comprises a plurality of resistors, at least two resistors of the plurality of resistors are connected in parallel in the first circuit configuration, and the plurality of resistors is part of a bridge circuit or a half bridge circuit in the second circuit configuration.

Aspect 3 is a thermal conductivity sensor of Aspect 1 or 2, further comprising: switching means configured to switch the measurement circuit between the first circuit configuration and the second circuit configuration.

Aspect 4 is a thermal conductivity sensor of Aspect 2 and Aspect 3, wherein the switching means are configured to switch at least two resistors of the plurality of resistors between a parallel connection of the at least two resistors and a serial connection of the at least two resistors.

Aspect 5 is a thermal conductivity sensor of one of the preceding Aspects, further comprising: a component configured to measure a voltage difference across the measurement circuit in the second circuit configuration, wherein the voltage difference is indicative of a thermal conductivity of an analysis gas.

Aspect 6 is a thermal conductivity sensor of one of the preceding Aspects, wherein: the measurement circuit comprises four resistors, the four resistors are connected in parallel in the first circuit configuration, and the four resistors are part of a Wheatstone bridge circuit in the second circuit configuration.

Aspect 7 is a thermal conductivity sensor of Aspect 6, wherein: a first resistor and a second resistor of the four resistors are configured to be exposed to an analysis gas, and a third resistor and a fourth resistor of the four resistors are exposed to a reference gas.

Aspect 8 is a thermal conductivity sensor of Aspect 3 and Aspect 7, wherein the switching means comprise: a first switch configured to connect a first node arranged between the first resistor and the fourth resistor to a ground potential and/or to disconnect the first node from the ground potential, a second switch configured to connect a second node arranged between the second resistor and the third resistor to a ground potential and/or to disconnect the second node from the ground potential, and at least one third switch configured to selectively connect a third node arranged between the second resistor and the fourth resistor to a ground potential or the supply voltage.

Aspect 9 is a thermal conductivity sensor of Aspect 8, wherein: in the first circuit configuration the first node is connected to the ground potential, the second node is connected to the ground potential, and the third node is connected to the supply voltage, and in the second circuit configuration the first node is disconnected from the ground potential, the second node is disconnected from the ground potential, and the third node is connected to the ground potential.

Aspect 10 is a thermal conductivity sensor of one of Aspects 6 to 9, wherein the first power dissipation is about four times higher than the first power dissipation.

Aspect 11 is a method for operating a thermal conductivity sensor, the method comprising: applying a supply voltage to a measurement circuit of the thermal conductivity sensor; performing a heating phase of the measurement circuit in a first circuit configuration of the measurement circuit, wherein the first circuit configuration is associated with a first power dissipation of the measurement circuit at the supply voltage; and performing a measurement phase of the measurement circuit in a second circuit configuration of the measurement circuit, wherein the second circuit configuration is associated with a second power dissipation of the measurement circuit at the supply voltage, wherein the first power dissipation is greater than the second power dissipation.

Aspect 12 is a method of Aspect 11, further comprising: switching the measurement circuit from the first circuit configuration to the second circuit configuration when the measurement circuit has reached a substantially stable temperature.

Aspect 13 is a method of Aspect 12, wherein switching the measurement circuit from the first circuit configuration to the second circuit configuration triggers performing a measurement for obtaining a measurement value.

Aspect 14 is a method of Aspect 13, wherein performing the measurement comprises measuring a voltage difference across the measurement circuit in the second circuit configuration.

Aspect 15 is a method of Aspect 14, further comprising: detecting an analysis gas and/or a concentration of an analysis gas based on the measurement value.

Aspect 16 is a method of one of Aspects 11 to 15, wherein the supply voltage is kept substantially constant during the heating phase and the measurement phase.

While this implementation has been described with reference to illustrative Aspects, this description is not intended to be construed in a limiting sense. Various modifications and combinations of the illustrative Aspects, as well as other Aspects of the implementation, will be apparent to persons skilled in the art upon reference to the description. It is therefore intended that the appended claims encompass any such modifications or Aspects.

1. Thermal conductivity sensor, comprising:
a measurement circuit configured to operate in a first circuit configuration during a heating phase of the measurement circuit and in a second circuit configuration during a measurement phase of the measurement circuit, wherein:
the first circuit configuration is associated with a first power dissipation of the measurement circuit at a supply voltage,
the second circuit configuration is associated with a second power dissipation of the measurement circuit at the supply voltage, and
the first power dissipation is greater than the second power dissipation.

2. The thermal conductivity sensor of claim 1, wherein:
the measurement circuit comprises a plurality of resistors,
at least two resistors of the plurality of resistors are connected in parallel in the first circuit configuration, and
the plurality of resistors is part of a bridge circuit or a half bridge circuit in the second circuit configuration.

3. The thermal conductivity sensor of claim 1, further comprising:
a switching component means configured to switch the measurement circuit between the first circuit configuration and the second circuit configuration.

4. The thermal conductivity sensor of claim 3, wherein the switching means-component is configured to switch at least two resistors of the plurality of resistors between a parallel connection of the at least two resistors and a serial connection of the at least two resistors.

5. The thermal conductivity sensor of claim 1, further comprising:
a component configured to measure a voltage difference across the measurement circuit in the second circuit configuration, wherein the voltage difference is indicative of a thermal conductivity of an analysis gas.

6. The thermal conductivity sensor of claim 1, wherein:
the measurement circuit comprises four resistors,
the four resistors are connected in parallel in the first circuit configuration, and
the four resistors are part of a Wheatstone bridge circuit in the second circuit configuration.

7. The thermal conductivity sensor of claim 6, wherein:
a first resistor and a second resistor of the four resistors are configured to be exposed to an analysis gas, and
a third resistor and a fourth resistor of the four resistors are exposed to a reference gas.

8. The thermal conductivity sensor of claim 7, wherein the switching means component comprises comprise:
a first switch configured to connect a first node arranged between the first resistor and the fourth resistor to a ground potential and/or to disconnect the first node from the ground potential,
a second switch configured to connect a second node arranged between the second resistor and the third resistor to the ground potential and/or to disconnect the second node from the ground potential, and
at least one third switch configured to selectively connect a third node arranged between the second resistor and the fourth resistor to the ground potential or the supply voltage.

9. The thermal conductivity sensor of claim 8, wherein:
in the first circuit configuration the first node is connected to the ground potential, the second node is connected to the ground potential, and the third node is connected to the supply voltage, and
in the second circuit configuration the first node is disconnected from the ground potential, the second node is disconnected from the ground potential, and the third node is connected to the ground potential.

10. The thermal conductivity sensor of claim 6, wherein the first power dissipation is four times higher than the first power dissipation.

11. A method for operating a thermal conductivity sensor, the method comprising:
applying a supply voltage to a measurement circuit of the thermal conductivity sensor;
performing a heating phase of the measurement circuit in a first circuit configuration of the measurement circuit, wherein the first circuit configuration is associated with a first power dissipation of the measurement circuit at the supply voltage; and
performing a measurement phase of the measurement circuit in a second circuit configuration of the measurement circuit, wherein the second circuit configuration is associated with a second power dissipation of the measurement circuit at the supply voltage,
wherein the first power dissipation is greater than the second power dissipation.

12. The method of claim 11, further comprising:
switching the measurement circuit from the first circuit configuration to the second circuit configuration when the measurement circuit has reached a substantially stable temperature.

13. The method of claim 12, wherein switching the measurement circuit from the first circuit configuration to the second circuit configuration triggers performing a measurement for obtaining a measurement value.

14. The method of claim 13, wherein performing the measurement comprises measuring a voltage difference across the measurement circuit in the second circuit configuration.

15. The method of claim 14, further comprising:
detecting an analysis gas and/or a concentration of the analysis gas based on the measurement value.

16. The method of claim 11, wherein the supply voltage is kept substantially constant during the heating phase and the measurement phase.

17. The method of claim 11, wherein the first power dissipation is four times higher than the first power dissipation.

18. The method of claim 11, further comprising:
connecting at least two resistors, of a plurality of resistors, in parallel to configure the measurement circuit in the first circuit configuration.

19. The method of claim 11, wherein at least two resistors, of a plurality of resistors, are included in a bridge circuit or a half bridge circuit in the second circuit configuration.

20. The method of claim 11, further comprising:
switching at least two resistors, of a plurality of resistors, between a parallel connection of the at least two resistors and a serial connection of the at least two resistors to switch between the first circuit configuration and the second circuit configuration.
